(19) United States
(12) Patent Application Publication
(10) Pub. No.: US 2026/0363940 A1
(43) Pub. Date: Nov. 19, 2026

(54) HANDHELD DEVICE FOR PROVIDING FLUIDIC MASSAGE

(71) Applicant: Shenzhen Kaiyan Medical Equipment Co., Ltd., Shenzhen (CN)

(72) Inventor: Alain Dijkstra, Amstelveen (NL)

(73) Assignee: Shenzhen Kaiyan Medical Equipment Co., Ltd., Shenzhen (CN)

(21) Appl. No.: 19/737,849

(22) Filed: Jul. 10, 2026

Related U.S. Application Data
(63) Continuation-in-part of application No. 18/047,291, filed on Oct. 18, 2022, now Pat. No. 12,734,096.

(30) Foreign Application Priority Data
Sep. 20, 2022 (CN) 202222478997.4

(51) Int. Cl. A61H 9/00; A61N 1/36

(52) U.S. Cl. CPC A61H 9/0021; A61H 2009/0042; A61H 2201/0107; A61H 2201/0153; A61H 2201/02; A61H 2201/10; A61H 2201/1238; A61H 2201/1607; A61H 2201/165; A61H 2201/1654; A61H 2201/169; A61H 2201/5002; A61N 1/36014

(57) ABSTRACT

A device for providing fluidic massage comprises a storage container adapted to store a fluid, a plurality of fluidic channels including a supply channel and a suction channel, at least one pump in fluidic communication with the storage container through the plurality of fluidic channels, one or more nozzles configured to impinge fluid flow in one or more fluid patterns on an inner surface of a bladder to provide fluidic massage to a user through the bladder, and a bladder encapsulating the one or more nozzles, wherein the bladder is adapted to be in contact with the user at an outer surface of the bladder. The device may be configured as a handheld device with a detachable bladder assembly, a wearable therapy pad, or a ball-shaped fluidic massage device.

### CROSS-REFERENCE TO RELATED APPLICATION

This application is a continuation-in-part of U.S. patent application Ser. No. 18/047,291, filed: Oct 18, 2022, entitled “HANDHELD DEVICE FOR PROVIDING FLUIDIC MASSAGE, the entire contents of which are incorporated by reference herein”.

### TECHNICAL FIELD

The present invention generally relates to therapeutic devices. More specifically the present invention relates to non-invasive therapeutic devices which may also be used for 18ecreational purposes.

### BACKGROUND ART

Many people suffer from joint pain, muscle aches, and inflammation. Muscle aches are also known as myalgia and can be felt in any area of the body with muscles. Depending on the cause, the discomfort may be mild or extremely severe. Muscle aches can occur in adults and children. Body pains associated with muscles affect individuals’ day-to-day activities and cause problems during movement. People with joint pain, inflammation, and damage to weight-bearing joints (i.e., hips, knees, ankles, feet, shoulders) caused by arthritis have mobility issues that affect their ability to work and perform common daily tasks. It can cause an inability to enjoy leisure activities, strained relationships, and problems at work.

Various devices have been known in the prior art that are used for body massages, exercise recovery massages, facial massages, and skin treatments for alleviating the pain of muscles and enhancing the skin condition. However, these devices are available in various shapes and sizes for different body parts are having significant structural and functional shortcomings. Presently, most of the known massage devices are provided with a motor connected to the massage head and a power source. These types of devices use mechanical energy to provide massage therapy by rotating the massage head in a circular motion or by moving the massage head in a linear motion. Such techniques can be quite uncomfortable and painful for the user as they can cause physical harm due to the nature of therapy and also it can create muscle spasms if not properly handled. Some devices provide water massage therapy like a water massage bed. The structural shortcoming of these devices is that either they are available in huge sizes, or they are available for a specific body part. For example, water massage beds are used for whole-body massage, but these are neither convenient nor cost-effective.

Therefore, there is a need for a handheld massage device that overcomes the disadvantages and limitations associated with the prior art and provides a more satisfactory solution.

### OBJECTS OF THE INVENTION

Some of the objects of the invention are as follows:

An object of the invention is to provide a handheld device that allows fluidic massage to a user on an affected part of the body;
Another object of the invention is that the handheld device be portable and ergonomically easy to use;
Yet another object of the invention is that the handheld device be adjustable and reconfigurable for varying portions of the body of the user and varying needs for the intensity of the fluidic massage; and
Yet another object of the invention is that the handheld device be non-invasive and ergonomically relatable for the user.

### SUMMARY OF THE INVENTION

According to a first aspect of the present invention, there is provided a handheld device for providing fluidic massage, the handheld device comprising a storage container adapted to store a fluid, one or more fluidic channels extending into the storage container, the one or more fluidic channels including one or more respective first ends and one or more respective second ends, wherein the one or more first ends are within the storage container, one or more pumps in fluidic communication with the storage container through the one or more fluidic channels, one or more nozzles provided at the one or more second ends of the fluidic channels, and a bladder encapsulating the one or more nozzles, wherein the bladder is adapted to be in contact with a user at an outer surface of the bladder, and receive fluid flow from the one or more nozzles, at an inner surface of the bladder.

In one embodiment of the invention, the handheld device further comprises a nozzle cap configured to modify flow characteristics of the fluid flow from the one or more nozzles.

In one embodiment of the invention, the nozzle cap includes one or more through holes of varying cross-sections.

In one embodiment of the invention, the nozzle cap is configured to be adjusted manually.

In one embodiment of the invention, the nozzle cap is configured to be controlled electronically.

In one embodiment of the invention, at least one of the one or more fluidic channels is a supply channel and at least one of the one or more fluidic channels is a suction channel.

In one embodiment of the invention, at least one of the one or more pumps is a supply pump and at least one of the one or more pumps is a suction pump.

In one embodiment of the invention, the handheld device further comprises a radiation plate within the space encapsulated by the bladder, the radiation plate including one or more radiation sources for emitting electromagnetic radiation.

In one embodiment of the invention, the one or more radiation sources includes one or more light emitting diodes.

In one embodiment of the invention, the handheld device further comprises a main control unit adapted to modify flow characteristics of the fluid flow from the one or more nozzles.

In one embodiment of the invention, the main control unit is configured to modify the flow characteristics through electronic adjustment of a nozzle cap.

In one embodiment of the invention, the main control unit is configured to modify the flow characteristics through electronic control of the one or more pumps.

In one embodiment of the invention, the bladder is made of one or more of a flexible polymer material and a fabric material.

In one embodiment of the invention, the flexible polymer material is selected from a group consisting of Polyvinyl Chloride (PVC), Silicone and Latex.

According to a second aspect of the present invention, there is provided a device for providing fluidic massage, the device comprising a housing, a storage container adapted to store a fluid, a plurality of fluidic channels including a supply channel and a suction channel, at least one pump in fluidic communication with the storage container through the plurality of fluidic channels, a main control unit configured to control operation of the at least one pump, a detachable bladder assembly configured to be removably coupled to the housing, the detachable bladder assembly comprising a bladder secured to an attachment rim, the attachment rim configured to releasably engage the housing, one or more nozzles positioned within the bladder when the detachable bladder assembly is coupled to the housing, the one or more nozzles configured to impinge fluid flow in one or more fluid patterns on an inner surface of the bladder to provide fluidic massage to a user through the bladder, wherein an outer surface of the bladder is adapted to be in contact with the user, and wherein the device is configured to simultaneously supply fluid to the bladder through the supply channel and suck fluid from the bladder through the suction channel to provide continuous fluid recirculation during the fluidic massage.

In one embodiment of the invention, the device is configured to initially supply fluid to the bladder through the supply channel until a predetermined volume of fluid accumulates within the bladder prior to initiating suction through the suction channel.

In one embodiment of the invention, the device is configured as a wearable therapy pad comprising a flexible pad body adapted to be removably worn over a selected portion of the body of the user, one or more fastening members configured to removably secure the wearable therapy pad around the selected body portion, and a plurality of bladders distributed across a body-contacting surface of the flexible pad body, each bladder encapsulating one or more nozzles.

According to a third aspect of the present invention, there is provided a ball-shaped fluidic massage device comprising a ball-shaped body having an inner layer and an outer layer, wherein the inner layer houses a storage container adapted to store a fluid, at least one pump, a main control unit, and a plurality of fluidic channels including a supply channel and a suction channel, wherein the outer layer comprises a bladder affixed to an exterior of the inner layer, the bladder having an outer surface adapted to contact a body of a user and an inner surface, one or more nozzles configured to impinge fluid flow in one or more fluid patterns on the inner surface of the bladder to provide fluidic massage to the user through the bladder, and wherein the device is configured to simultaneously supply fluid to the bladder through the supply channel and suck fluid from the bladder through the suction channel to provide continuous fluid recirculation during the fluidic massage.

According to a fourth aspect of the present invention, there is provided a method for providing fluidic massage to a user, the method comprising positioning a bladder in contact with a body of the user, wherein an outer surface of the bladder contacts the body and one or more nozzles are encapsulated within the bladder, supplying fluid from a storage container through a supply channel to the one or more nozzles, impinging the fluid from the one or more nozzles in one or more fluid patterns onto an inner surface of the bladder to provide fluidic massage to the user through the bladder, simultaneously sucking fluid from the bladder through a suction channel, and returning the fluid to the storage container to provide continuous fluid recirculation during the fluidic massage.

In the context of the specification, the term “processor” refers to one or more of microprocessors, a microcontroller, a general-purpose processor, a Field Programmable Gate Array (FPGA), or an Application Specific Integrated Circuit (ASIC), and the like.

In the context of the specification, the phrase “storage memory” refers to one or more of a volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc., or a non-volatile storage memory such as EPROM, EEPROM or flash memory or the like.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelength, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a handheld device for providing fluidic massage, according to an embodiment of the present invention;

FIG. 1B illustrates a sectional view of the handheld device of FIG. 1A;

FIG. 2A illustrates several nozzles covered by a nozzle cap, in accordance with a second embodiment of the present invention;

FIG. 2B illustrates a partial sectional view of the handheld device of FIG. 2A;

FIG. 3 illustrates a radiation plate, in accordance with a third embodiment of the present invention;

FIG. 4 illustrates the handheld device, in accordance with a fourth embodiment of the present invention;

FIG. 5 illustrates a user applying fluidic massage through the handheld device, in accordance with an embodiment of the present invention;

FIGS. 6A and 6B illustrates two distinct fluid flow patterns emanating from a nozzle, in accordance with an embodiment of the present invention; and

FIG. 7 illustrates a partial sectional view of the handheld device, in accordance with yet another embodiment of the present invention.

FIG. 8 illustrates an exploded view of a wearable therapy pad, in accordance with an embodiment of the present invention;

FIG. 9 illustrates a cross-sectional view of a wearable therapy cap, in accordance with an embodiment of the present invention;

FIG. 10 illustrates a cross-sectional view of a ball-shaped fluidic massage device, in accordance with an embodiment of the present invention;

FIG. 11 illustrates a bladder configuration comprising a contact portion and rigid side walls, in accordance with an embodiment of the present invention; and

FIG. 12 illustrates a flowchart depicting a method for providing fluidic massage to a user, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

It is envisaged that a handheld device that is ergonomically usable, for example in the shape of a massage gun, be provided that delivers fluidic massage to several body portions of a user. The handheld device is envisaged to include a storage container and fluid channels that deliver the fluid to and from the storage container. The fluid supply and return are envisaged to be aided through one or more fluid pumps. Nozzles are to be attached to the fluidic channels that would impinge the fluid in several fluid patterns on an inner surface of a bladder encapsulating the nozzles. While the outer surface of the bladder is envisaged to be in contact with the body of the user, to ensure that the fluid does not come in direct contact with the body of the user.

Flow characteristics of the fluid flow may be altered to provide fluidic massages for varying body portions and varying intensities by manual or electronic control of the pumps and/or by varying cross-sections and patterns of the fluid from the nozzles. Additional adjustments are also envisaged to be provided following user inputs received through push buttons or touch interfaces. It is further envisaged that the fluid may be water, a solution with organic or inorganic solvent, an emulsion or essential oils, or the like. Referring to the figures, the invention will now be described in further detail.

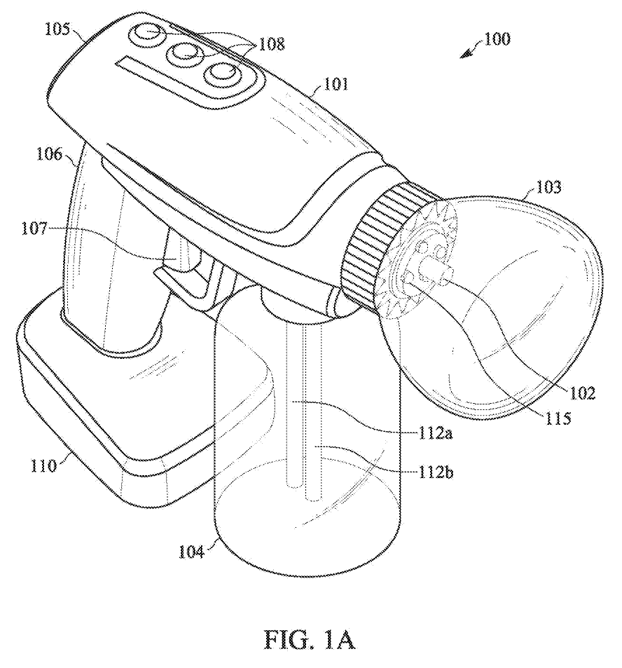

FIG. 1A illustrates a perspective view of a handheld device 100 (hereinafter referred to as “the device”) for providing fluidic massage, according to an embodiment of the present invention. The device 100 includes a housing 101 for encapsulating several different elements of the device 100. The housing 101 may be made from metal, plastic material or composite materials, etc. The housing 101 allows the device 100 to be structured as a massage gun providing relatable ergonomics to the user. The device 100 further includes a nozzle 102 configured to impinge fluid flow on an inner surface of a bladder 103. The bladder 103 is adapted to encapsulate the nozzle 102. During operation of the device 100 the bladder 103 is adapted to be in contact with a user at an outer surface of the bladder 103, and receive fluid flow from the nozzle 102, at an inner surface of the bladder 103. The bladder 103 may be made of one or more of a flexible polymer material and a fabric material. Also, the flexible polymer material may be selected from a group consisting of Polyvinyl Chloride (PVC), Silicone, and Latex

Further attached with the housing 101 is a storage container 104. The storage container 104 is adapted to store a fluid. The device 100 also includes a display unit 105 provided on the rear end of the device 100 and adapted to be facing the user. In that regard, the display unit 105 may be an LCD or LED based display unit with or without touch input capability. The input may also be provided through a keyboard. Further illustrated in FIG. 1A is a trigger element 107. The trigger element 107 acts as a switch allowing activation and deactivation of the fluidic massage, when pressed and released, respectively. To electronically modify fluid flow characteristics, such as speed, intensity, pulsating or continuous application, direction, and flow pattern, of the fluidic massage, the device 100 may also be provided with several push buttons 108 for receiving several inputs from the user. A base structure 110 allows the device 100 to be located upon a flat surface when device 100 is not being utilized.

Further illustrated in FIG. 1A are fluidic channels 112a and 112b. The fluidic channels 112a and 112b may be structured as cavities in the housing 101, or the cavities may be provided with tubular inserts acting as fluidic channels 112a and 112b. The fluidic channels 112a and 112b have respective first ends that are located within the storage container 104. Also, the respective second ends of the fluidic channels 112a and 112b are connected with the nozzle 102. In that regard, the fluidic channel 112a may act as a fluid supply channel, and the fluidic channel 112b may act as a suction channel or vice versa. In several embodiments of the present invention, the device 100 may include several more fluidic channels, in a manner that at least one of the fluidic channels is a supply channel and at least one of fluidic channels is a suction channel. FIG. 1A also illustrates radiation sources 207. In several embodiments of the present invention, the radiation sources 207 may include Light Emitting Diodes (LEDs).

The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

FIG. 1B illustrates a sectional view of the device 100 of FIG. 1A. FIG. 1B illustrates a pump 114. The pump 114 is in fluidic communication with the storage container 104 through the fluidic channels 112a and 112b. It is further illustrated that the pump 114 doubles as both a supply pump and a suction pump. Also, the fluidic channel 112a acts as a suction channel and the fluidic channel 112b acts as supply channel. However, in several embodiments of the invention, the device 100 may include more than one pumps. In such a configuration, at least one pump may act as a supply pump and at least one pump may act as a suction pump. FIG. 1B illustrates a main control unit 111 including a processor 111b and a storage memory 111a. The main control unit 111 is adapted to modify the flow characteristics of the fluid flow from the nozzle 102. The main control unit 111 may either act automatically based on machine-readable instructions stored in the storage memory 111a and/or in response to inputs received through touch interface of the display unit 105 and/or the push buttons 108. A battery 109 has been provided within the housing 101 to power, inter alia, the pump 114 and the main control unit 111. In several alternate embodiments, the device 100 may operate through power derived from an AC or DC power supply.

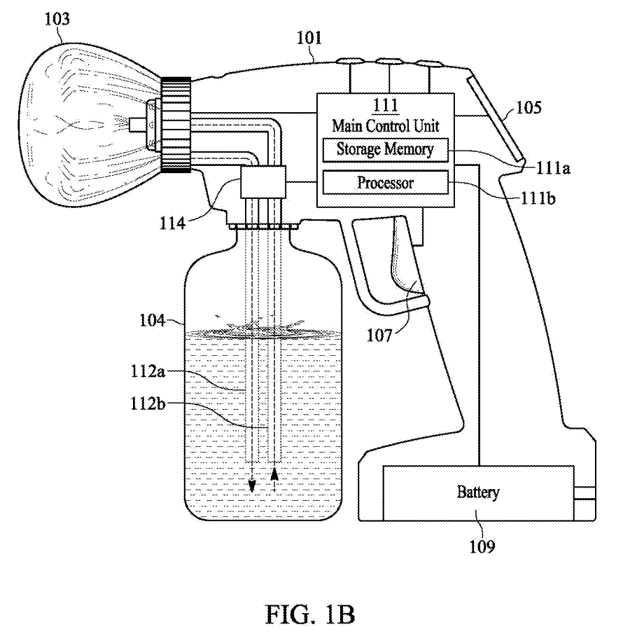

FIG. 2A illustrates several nozzles 201a, 201b, 201c, 201d, 201e, and 201f covered by a nozzle cap 203, in accordance with a second embodiment 200 of the present invention. The nozzle cap 203 is configured to modify the flow characteristic of the fluid flow from the nozzles 201a, 201b, 201c, 201d, 201e, and 201f. FIG. 2B illustrates a partial sectional view of the device 100 of FIG. 2A. It is further to be noted that the nozzle cap 203 is located over the nozzles 201a, 201b, 201c, 201d, 201e, and 201f and within the space encapsulated by the bladder 103. The nozzle cap 203 may include several through holes 204 of varying cross-sections, such as a rectangular cross-section 202 illustrated in FIG. 2. Other cross-sections such as circular, oval, and triangular are also illustrated in FIG. 2. The varying cross-section will cause the fluid flow impinging upon the inner surface of the bladder 103 to take several different flow patterns, as will be illustrated later in the specification.

The nozzle cap 203 may be adjusted 206 manually through rotation of the rotating element 208 with knurling provided on an external surface of the rotating element 208. The nozzle cap 203 may also be adjusted electronically through the main control unit 111 via an electrical motor 210 located within the housing 101, in accordance with the instructions stored in the storage memory 111a and/or inputs received through the display unit 105 and/or the push buttons 108. In several alternate embodiments, the main control unit 111 may be configured to modify the flow characteristics through electronic control of the one or more pumps 114. In that manner, the pumps 114 may be operated in several distinct modes such as pulsating and continuous or at varying speeds to modify the flow characteristics of the fluid flow. In several embodiments of the invention, the nozzle cap 203 may also double as an opaque or a diaphanous cover for a radiation source 207 illustrated in FIG. 2.

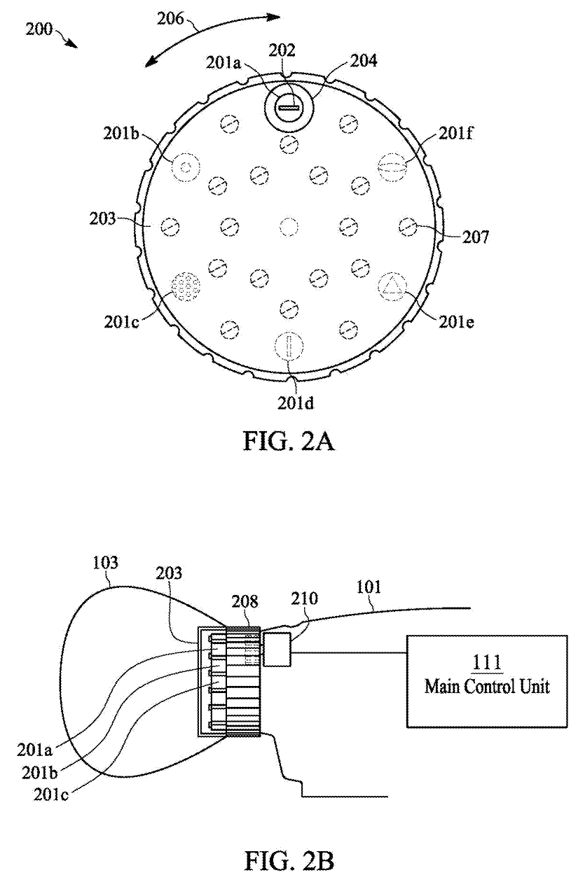

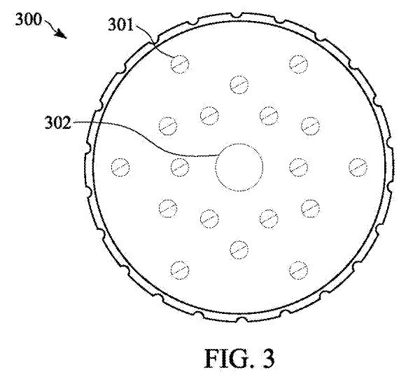

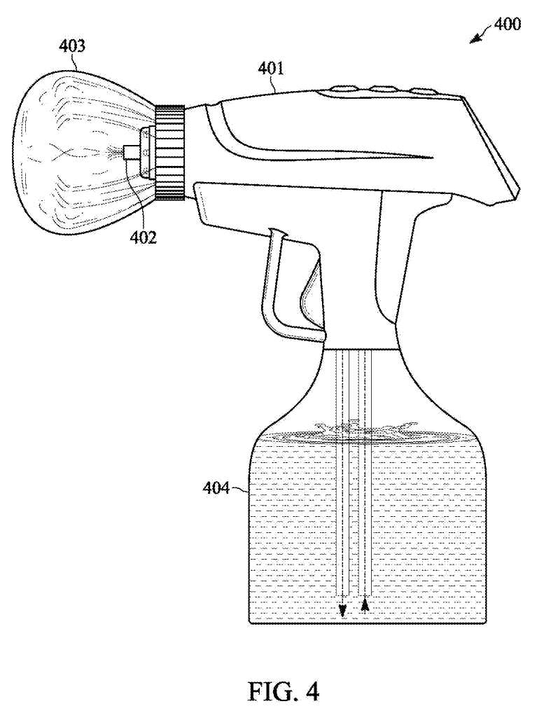

FIG. 3 illustrates a radiation plate 300, in accordance with a third embodiment of the present invention. The radiation plate 300 is located within the space encapsulated by the bladder 103 and includes several radiation sources 301. Further, a nozzle 302 is located at the center of the radiation plate 300. The radiation sources 301 may be provided in several distinct patterns on the radiation plate 300 for emitting therapeutic electromagnetic radiation. FIG. 4 illustrates the device 100, in accordance with a fourth embodiment 400 of the present invention. The embodiment 400 illustrates the housing 401, the nozzle 402, the bladder 403, and the storage container 404 located in place of the base structure 110. FIG. 5 illustrates a user 502 applying a fluidic massage through the device 501 on biceps portion of their body, in accordance with an embodiment 500 of the present invention. The device 501 in that manner may be held in several different orientations for providing fluidic massage at several different portions of the body of the user 502.

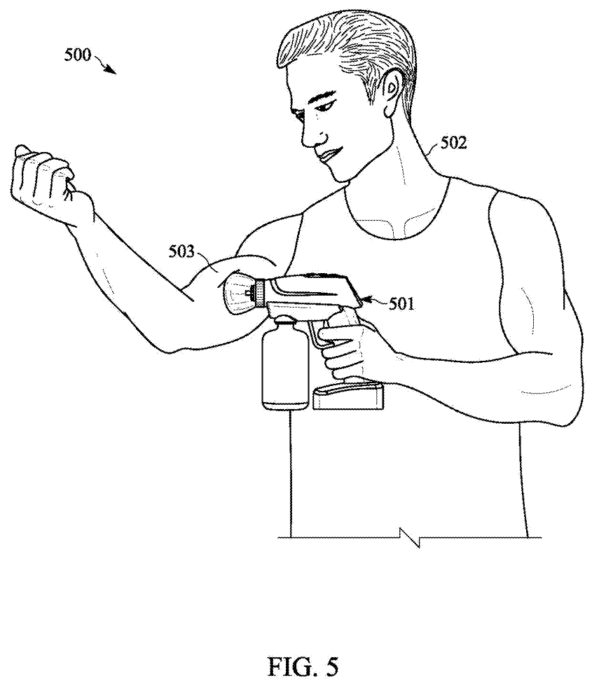

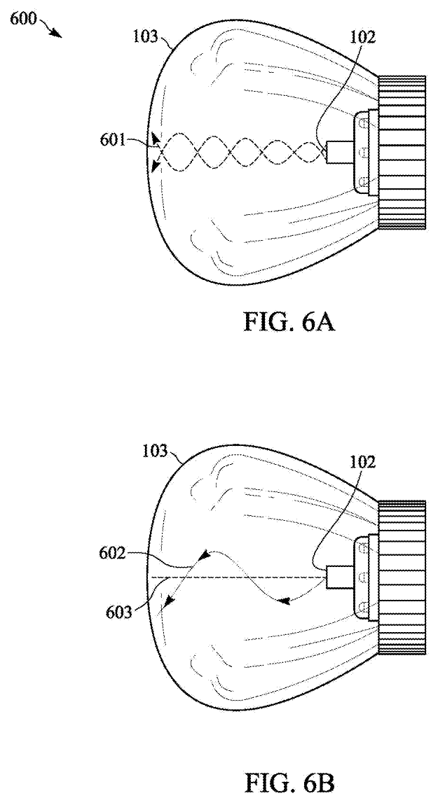

FIGS. 6A and 6B illustrates two distinct fluid flow patterns 601 and 602, emanating from the nozzle 102, in accordance with an embodiment 600 of the present invention. The distinct flow patterns include a double flow pattern 601 and a sinusoidal pattern 602 (about an axis 603) and may be achieved though combinations of adjustment 206 of the nozzle cap 203 and varying modes of operation and speeds of the pump 114. FIG. 7 illustrates a partial sectional view of the device 100, in accordance with yet another embodiment 700 of the present invention. In the embodiment 700, the pump 114 acts both as a supply pump and a suction pump and the fluid 701 is drawn into a suction port 702 provided at the second end of the fluidic channel 112a.

In use, the user 502 may bring the bladder 103 or 403 in contact with a portion of their body and apply pressure on the trigger element 107. The trigger element 107 would act as a switch thereby activating the main control unit 111 and the pump 114. The fluid will be supplied to the nozzle 102 though the fluidic channel 112b. The nozzle 102 would impinge the fluid onto the inner surface of the bladder 103 or 403 thereby providing fluidic massage to the user 502. The excess fluid in the bladder would be sucked through the suction port 702 and returned to the storage container 104 via the fluidic channel 112a. The flow characteristics of the fluid flow may be adjusted by the user 502 through touch interface of the display unit 105 or through the push buttons 108. Further, the user 502 may also manually rotate the nozzle cap 203 to change the fluid flow pattern and intensity etc.

In various embodiments of the present invention, the device may be configured to initially supply fluid to the bladder 103 through the supply channel (fluidic channel 112a) until a predetermined volume of fluid accumulates within the bladder 103 prior to initiating suction through the suction channel (fluidic channel 112b). Once the predetermined volume has accumulated, the device may simultaneously supply additional fluid while withdrawing excess fluid, thereby maintaining a substantially constant fluid volume within the bladder 103 during therapeutic operation. This operational sequence may be applied to any of the embodiments described herein, including handheld devices, wearable therapy pads, wearable therapy caps, ball-shaped devices, or other configurations.

In various embodiments of the present invention, the bladder 103 may be integrated with the housing 101 as a detachable bladder assembly configured to be removably coupled to the device. The detachable bladder assembly may comprise a bladder 103 secured to an attachment rim, the attachment rim configured to releasably engage the housing through one or more locking mechanisms including threaded engagement, bayonet engagement, snap-fit engagement, magnetic coupling, latch mechanisms, or quarter-turn connectors. The detachable bladder assembly may be interchangeable with a plurality of different bladder assemblies having different diameters, heights, contours, flexibility, wall thicknesses, internal volumes, or contact surface geometries for treating different portions of a user's body. The detachable configuration enables replacement of the bladder 103 after prolonged use or for hygienic purposes without replacing the remaining components of the device.

In various embodiments of the present invention, one or more radiation sources 207 may be provided on or integrated with the bladder 103 rather than being positioned within the space encapsulated by the bladder 103. The one or more radiation sources 207 may be arranged as a radiation ring, radiation plate, flexible light panel, light strip, or one or more individual light-emitting elements coupled to an outer surface, an inner surface, or embedded within a wall of the bladder. The bladder 103 may be fabricated from a flexible transparent, translucent, or optically transmissive material, or may include one or more transparent or translucent regions through which electromagnetic radiation emitted by the radiation sources 207 passes toward the body of the user. This configuration enables concurrent phototherapy and fluidic massage treatment in any of the embodiments described herein.

In various embodiments of the present invention, the storage container 104 may be positioned externally of the device 100 and fluidically connected thereto through one or more flexible conduits. The external positioning of the storage container enables the massage-delivering portion of the device 100 to remain lightweight and flexible while allowing the storage container to be conveniently removed, refilled, replaced, or cleaned without removing or disassembling the massage-delivering portion. The storage container 104 may be worn on a waist belt, shoulder harness, garment, backpack, carried by the user, or positioned adjacent to the user.

In various embodiments of the present invention, the main control unit 111 may be configured to selectively adjust one or more operational parameters including flow rate, suction pressure, pulsation frequency, massage intensity, treatment duration, circulation sequence, intermittent operation, continuous operation, or combinations thereof. The main control unit 111 may adjust these parameters in response to user inputs received through one or more push buttons, a touch interface, a wireless communication module, or other input mechanisms. Different groups of bladders may be activated independently or simultaneously to create predetermined massage patterns.

In various embodiments of the present invention, the device 100 is not limited to a handheld configuration. The device 100 may be configured as a wearable therapy pad adapted to be removably worn over a selected portion of the body, a wearable therapy cap adapted to surround at least a portion of the head, a ball-shaped device adapted to be rolled or pressed across body surfaces, a massage blanket comprising a flexible blanket body with a plurality of bladders distributed across a body-contacting surface adapted to cover a substantial portion or entirety of the user's body, a massage bed comprising a support surface with a plurality of bladders distributed across the surface for providing full-body fluidic massage while the user lies on the bed, a massage roller comprising a cylindrical body with an inner layer housing the storage container, the at least one pump, and the main control unit, and an outer layer comprising a bladder adapted to be rolled across body surfaces, a massage mat or mattress overlay configured to be placed on a bed, chair, or floor surface, a massage chair pad configured to be placed on a chair or seat for providing fluidic massage to a seated user, a massage cushion or pillow with one or more bladders for targeted fluidic massage to areas such as the neck or lumbar region, a massage glove comprising a glove-shaped body with one or more bladders distributed on palm and finger surfaces for delivering fluidic massage through hand contact, a massage sleeve comprising a tubular body configured to surround a limb of the user, a massage belt configured to be worn around the waist or torso of the user, a massage insole or foot pad configured for delivering fluidic massage to the foot, a facial massage device comprising a compact body with one or more small bladders adapted for facial massage and skincare treatment, or other configurations suitable for delivering fluidic massage to various body portions. The features described herein, including the detachable bladder assembly, transparent bladder with radiation sources, external storage container, predetermined fluid accumulation, and main control unit adjustments, may be applied individually or in combination to any of these device configurations.

In various embodiments of the present invention, the massage medium may comprise one or more of water, a therapeutic fluid, a solution with organic or inorganic solvent, an emulsion, essential oils, or air. When air is used as the massage medium, the device operates as a pneumatic massage system wherein pressurized air is supplied through the supply channel to one or more nozzles, impinged upon the inner surface of the bladder 103 to cause expansion and deformation of the bladder against the user's body, and simultaneously withdrawn through the suction channel to provide continuous air recirculation. The use of air as the massage medium may provide advantages including reduced weight, elimination of fluid leakage concerns, and simplified maintenance.

In various embodiments of the present invention, the bladder 103 may comprise a contact portion and one or more side walls having different material properties. The contact portion configured to contact the skin of the user may be fabricated from a flexible material including flexible polymer materials, elastomeric materials, silicone-based materials, or fabric materials to comfortably conform to the contour of the body portion being treated and effectively transmit the fluidic massage sensation. The one or more side walls may be fabricated from a substantially rigid or semi-rigid material, including polymeric materials, thermoplastic materials, or composite materials, to maintain the structural integrity of the bladder during operation and prevent the bladder from becoming flaccid when the massage medium is withdrawn through the suction channel. This configuration ensures consistent bladder geometry and massage performance throughout the treatment cycle.

In various embodiments of the present invention, the plurality of bladders in a wearable therapy pad or wearable therapy cap may be arranged in a plurality of distinct zones. The main control unit 111 may be configured to independently control massage medium supply and suction to each zone through dedicated fluidic channels or selectively actuated valves. This zone-based configuration enables the device to provide various massage patterns, including sequential activation wherein zones are activated one after another in a predetermined sequence, pulse-based massage wherein zones are rapidly activated and deactivated to create pulsating sensations, wave-like massage patterns wherein activation propagates across zones to simulate rolling or kneading motions, and localized massage wherein specific zones are activated to target particular body regions. The main control unit 111 may store a plurality of predetermined massage programs and enable user selection through push buttons, touch interface, or wireless communication module.

In various embodiments of the present invention, the device or system may further comprise a temperature control unit configured to heat or cool the massage medium before supply through the supply channel. The temperature control unit may include one or more heating elements such as resistive heaters, positive temperature coefficient heaters, or heat exchangers to warm the massage medium for thermotherapy applications, including muscle relaxation, pain relief, and improved circulation. The temperature control unit may alternatively or additionally include one or more cooling mechanisms such as thermoelectric coolers, Peltier elements, refrigerant-based cooling systems, or ice reservoirs to cool the massage medium for cryotherapy applications including inflammation reduction, swelling reduction, and acute injury treatment. A temperature sensor may be provided to monitor the temperature of the massage medium and enable the main control unit to maintain a user-selected temperature within a predetermined range.

In various embodiments of the present invention, the device or system may further comprise one or more electrical stimulation elements configured to deliver transcutaneous electrical nerve stimulation (TENS) or electrical muscle stimulation (EMS) to the user in conjunction with the fluidic massage. The electrical stimulation elements may include one or more electrodes positioned on or adjacent to the bladder, integrated within the bladder wall, or provided on a separate electrode pad configured to be positioned between the bladder and the user's skin. The main control unit may be configured to control the electrical stimulation parameters, including pulse frequency, pulse width, pulse amplitude, waveform shape, and treatment duration. The combination of fluidic massage with TENS may provide enhanced pain relief through gate control theory and endorphin release, while the combination with EMS may provide muscle strengthening, muscle re-education, and prevention of muscle atrophy in conjunction with the therapeutic massage.

In various embodiments of the present invention, the device 100 may be configured as a fluidic massage system comprising a massage delivery unit, a storage unit, a pump unit, and a control unit. The massage delivery unit comprises one or more bladders, each bladder 103 having an outer surface adapted to contact a body of a user and an inner surface, and one or more nozzles configured to impinge a massage medium in one or more patterns on the inner surface of the one or more bladders to provide fluidic massage to the user through the one or more bladders. The storage unit comprises a storage container adapted to store the massage medium. The pump unit comprises at least one pump configured to circulate the massage medium through a plurality of fluidic channels, including a supply channel and a suction channel fluidically connecting the storage unit, the pump unit, and the massage delivery unit. The control unit is configured to control the operation of the at least one pump. In various embodiments, the pump unit may be positioned internally to the massage delivery unit, or the pump unit may be positioned external to the massage delivery unit and fluidically connected thereto through one or more flexible conduits. Similarly, the storage unit may be positioned internal to the massage delivery unit, or the storage unit may be positioned external to the massage delivery unit and fluidically connected thereto through one or more flexible conduits. The system is configured to simultaneously supply the massage medium to the one or more bladders through the supply channel and suck the massage medium from the one or more bladders through the suction channel to provide continuous recirculation during the fluidic massage.

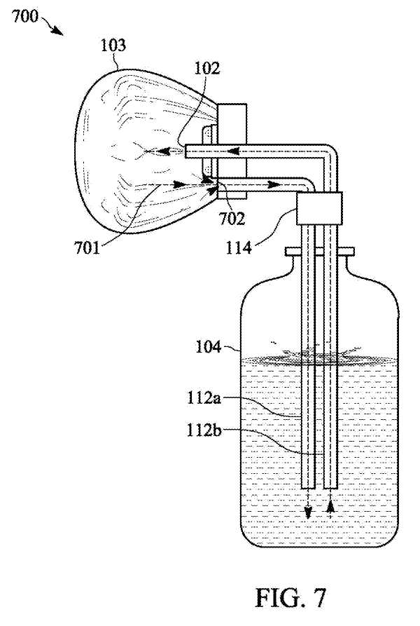

Referring to FIG. 8, in an alternate embodiment, the handheld device can be a wearable therapy pad 800. The wearable therapy pad 800 is configured as a flexible therapy pad adapted to be removably worn over a selected portion of the body of a user. In various embodiments, the wearable therapy pad 800 may be configured as a back pad, shoulder pad, neck pad, knee pad, elbow pad, wrist pad, ankle pad, thigh pad, calf pad, hip pad, abdominal pad, chest pad, foot pad, head pad, or any other therapeutic pad adapted to conform to a desired treatment region of the user's body. The therapy pad 800 includes a flexible pad body 801 formed from one or more flexible polymer materials, elastomeric materials, textile materials, or combinations thereof to comfortably conform to the contour of the body portion being treated. One or more fastening members 802, including straps, elastic bands, hook-and-loop fasteners, buckles, clips, sleeves, wraps, or similar securing mechanisms, may be provided to removably secure the therapy pad 800 around the selected body portion.

The flexible pad body 801 encapsulates a plurality of bladders 103 distributed across a body-contacting surface of the therapy pad 800. The plurality of bladders 103 are configured such that their outer surfaces contact the skin of the user while their inner surfaces receive fluid flow for generating fluidic massage. Each bladder 103 may encapsulate one or more nozzles 102, wherein the nozzles 102 are configured to discharge fluid toward the inner surface of the corresponding bladder. Consequently, the therapeutic fluid remains completely enclosed within the plurality of bladders 103 while deformation of the flexible bladder walls transmits localized pressure, pulsation, vibration, and fluidic massage to the underlying body tissue. The plurality of bladders 103 may be arranged in rows, columns, staggered arrays, concentric arrangements, honeycomb patterns, lattice arrangements, or any other suitable configuration depending upon the treatment region.

In the present embodiment, the storage container 104 is positioned externally of the therapy pad 800 and is fluidically connected thereto through one or more flexible conduits 804. The storage container 104 may be worn on a waist belt, shoulder harness, garment, backpack, carried by the user, positioned adjacent to the user, or otherwise supported independently from the therapy pad 800. Positioning the storage container 104 externally enables the therapy pad 800 to remain lightweight and flexible while allowing the storage container 104 to be conveniently removed, refilled, replaced, or cleaned without removing or disassembling the therapy pad 800. The storage container 104 stores the therapeutic fluid and is fluidically coupled to one or more pumps 114 through fluidic channels 112a and 112b. In one embodiment, the fluidic channel 112b functions as a supply channel for delivering fluid from the storage container 104 to the plurality of nozzles 102, while the fluidic channel 112a functions as a suction channel configured to withdraw the fluid from the plurality of bladders 103 and return the fluid back to the storage container 104, thereby establishing a substantially closed-loop fluid circulation system.

The wearable therapy pad 800 further includes a trigger element 107 electrically connected to the main control unit 111 through an elongated electrical cable 803. The elongated electrical cable 803 enables the trigger element 107 to be comfortably held and actuated by the user irrespective of the location of the therapy pad 800 on the body. Upon depression of the trigger element 107, the main control unit 111 activates the one or more pumps 114 to initiate circulation of the therapeutic fluid. Fluid is pumped from the storage container 104 through the supply channel (fluidic channel 112a) and discharged through the plurality of nozzles 102 into the respective bladders 103. The resulting fluid pressure causes localized expansion and deformation of the plurality of bladders 103 against the user's body, thereby producing a therapeutic fluidic massage. Simultaneously or subsequently, the therapeutic fluid is withdrawn from the plurality of bladders 103 through the fluidic channel 112b (suction channel) and returned to the storage container 104, thereby allowing continuous recirculation of the therapeutic fluid during operation.

The main control unit 111 may selectively control the operation of the one or more pumps 114 to generate various massage modes, including continuous flow, intermittent flow, pulsating flow, cyclic pressure variation, sequential fluid delivery, localized massage, or region-wise massage across the plurality of bladders 103. In various embodiments, different groups of the plurality of bladders 103 may be supplied with fluid independently or simultaneously to create predetermined massage patterns that propagate across the therapy pad 800. The wearable therapy pad 800 may further incorporate one or more radiation sources 207, heating elements, cooling elements, vibration generators, pressure sensors, or other therapeutic modules positioned within or adjacent to the plurality of bladders 103 to provide combined therapeutic treatment while maintaining the closed-loop fluidic massage system described herein.

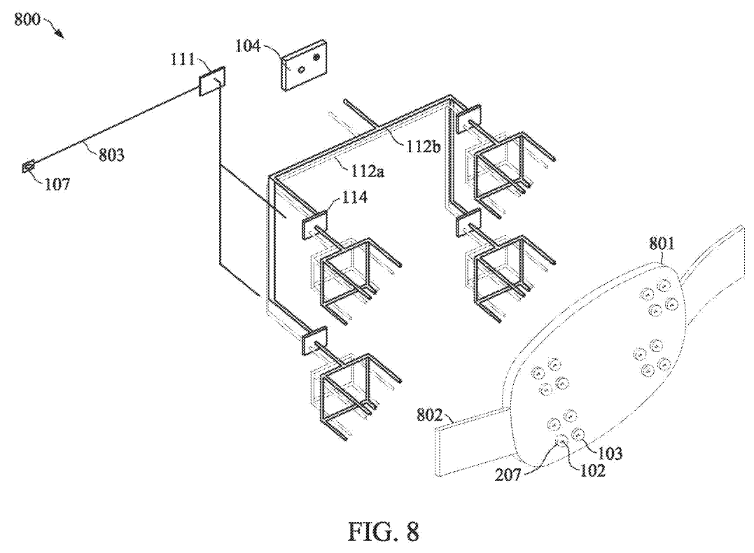

Referring to FIG. 9, in an alternate embodiment, the handheld device can be a wearable therapy cap 900. The wearable therapy cap 900 is adapted to surround at least a portion of the head of a user for delivering fluidic massage to the scalp. The therapy cap 900 comprises a flexible cap body 901 fabricated from one or more flexible polymer materials, elastomeric materials, fabric materials, or combinations thereof. The cap body 901 is configured to conform to the contour of the user's head and may include one or more adjustable fastening members, such as elastic bands, drawstrings, buckles, hook-and-loop fasteners, adjustable straps, or similar securing mechanisms, for accommodating different head sizes.

The cap body 901 encapsulates a plurality of bladders 103 distributed over an inner surface of the therapy cap 900 such that the outer surfaces of the plurality of bladders 103 contact the scalp of the user during use. Each bladder 103 may encapsulate one or more nozzles 102 configured to discharge therapeutic fluid toward the inner surface of the corresponding bladder. The plurality of bladders 103 may be arranged over the crown region, frontal region, temporal regions, occipital region, or substantially the entire inner surface of the therapy cap 900. The fluid discharged by the nozzles 102 repeatedly impinges upon the inner surfaces of the plurality of bladders 103, thereby causing localized expansion and deformation of the flexible bladders 103 to generate therapeutic fluidic massage over the scalp without permitting direct contact between the therapeutic fluid and the user's skin or hair.

A storage container 104 adapted to store a therapeutic fluid is positioned externally of the therapy cap 900 and is fluidically connected thereto through one or more flexible fluidic channels 112a and 112b. In various embodiments, the storage container 104 may be removably supported on a belt, waistband, shoulder strap, garment, portable carrying pouch, bedside support, chair, or another external support structure to facilitate convenient refilling and replacement of the therapeutic fluid while minimizing the weight carried by the therapy cap 900. The fluidic channel 112b functions as a supply channel for delivering therapeutic fluid from the storage container 104 to the plurality of nozzles 102, while the fluidic channel 112a functions as a suction channel for withdrawing the therapeutic fluid from the plurality of bladders 103 and returning the therapeutic fluid to the storage container 104. One or more pumps 114 establish continuous circulation of the therapeutic fluid through the closed-loop fluidic system.

The therapy cap 900 further comprises a trigger element 107 electrically coupled to the main control unit 111 through an elongated electrical cable 903. The elongated electrical cable 903 permits the trigger element 107 to be comfortably held and actuated by the user while the therapy cap 900 is worn. Upon activation of the trigger element 107, the main control unit 111 operates the one or more pumps 114 to pump therapeutic fluid from the storage container 104 through the supply channel (fluidic channel 112a) toward the plurality of nozzles 102. The therapeutic fluid impinges upon the inner surfaces of the plurality of bladders 103 to periodically expand the bladders 103 and apply fluidic massage to the scalp. Subsequently, the therapeutic fluid is withdrawn through the fluidic channel 112b (suction channel) and returned to the storage container 104 for continuous recirculation.

The main control unit 111 may selectively regulate one or more operational parameters, including fluid pressure, suction pressure, flow rate, pulsation frequency, treatment duration, circulation sequence, massage intensity, intermittent operation, continuous operation, or combinations thereof. In several embodiments, different groups of the plurality of bladders 103 may be activated independently or sequentially to generate wave-like, pulsating, kneading, rolling, or localized massage patterns across different regions of the scalp. The therapy cap 900 may further incorporate one or more radiation sources 207, heating elements, cooling elements, vibration elements, electrical stimulation elements, or combinations thereof within the cap body 901 to provide multimodal therapeutic treatment in conjunction with the fluidic massage generated by the plurality of bladders 103.

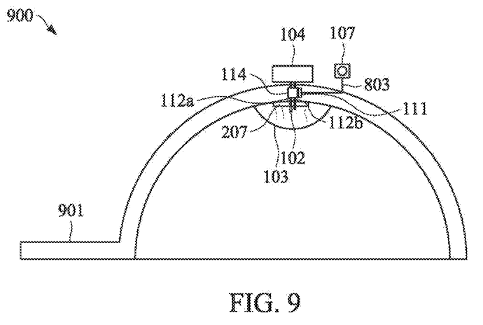

Referring to FIG. 10, in yet another embodiment, the fluidic massage device is configured as a ball-shaped fluidic massage device 1000. The ball-shaped fluidic massage device 1000 comprises a ball-shaped body having an inner layer 1001 and an outer layer 1002. The ball-shaped body may be substantially spherical, ovoid, ellipsoidal, or any other generally rounded configuration adapted to be rolled, pressed, or maneuvered across various portions of a user's body during therapeutic use. The ball-shaped configuration enables the user to apply fluidic massage to body portions that may be difficult to access with handheld or wearable embodiments, and permits massage application through sliding motions across curved body surfaces such as the back, shoulders, legs, and feet.

The inner layer 1001 houses a storage container 104 adapted to store a therapeutic fluid, at least one pump 114, a main control unit 111 including a processor 111b and a storage memory 111a, and a plurality of fluidic channels including a supply channel 112a and a suction channel 112b. The inner layer 1001 may be formed from a rigid or semi-rigid material including polymeric materials, thermoplastic materials, composite materials, or combinations thereof, providing structural support for the internal components while maintaining a substantially lightweight construction. A battery 109 may be housed within the inner layer 1001 to provide portable power to the at least one pump 114, the main control unit 111, and any additional electronic components. In alternative embodiments, the ball-shaped fluidic massage device 1000 may be configured to receive power through an external power cord or a wireless charging system.

The outer layer 1002 comprises a bladder 103 affixed to the exterior of the inner layer 1001. The bladder 103 has an outer surface adapted to contact the body of a user during therapeutic use and an inner surface configured to receive fluid flow from one or more nozzles 102. The bladder 103 may substantially surround the inner layer 1001, or may be provided over one or more selected regions of the inner layer 1001 corresponding to intended contact areas during massage application. The bladder 103 may be fabricated from one or more flexible polymer materials, elastomeric materials, silicone-based materials, or fabric materials, and may be permanently affixed to the inner layer 1001 through adhesive bonding, mechanical fastening, overmolding, heat sealing, or combinations thereof. In alternative embodiments, the bladder 103 may be removably coupled to the inner layer 1001 to facilitate cleaning, replacement, or interchanging of bladders having different surface textures, flexibility, or contact geometries.

One or more nozzles 102 are positioned at an interface between the inner layer 1001 and the outer layer 1002, configured to impinge fluid flow in one or more fluid patterns on the inner surface of the bladder 103 to provide fluidic massage to the user through the bladder 103. The one or more nozzles 102 are fluidically connected to the supply channel (fluidic channel 112a) for receiving therapeutic fluid from the storage container 104. A suction port 702 is provided in fluidic communication with the suction channel (fluidic channel 112b) for withdrawing excess fluid from within the bladder 103 and returning the fluid to the storage container 104.

During operation, the ball-shaped fluidic massage device 1000 is configured to simultaneously supply fluid to the bladder 103 through the supply channel 112a and suck fluid from the bladder 103 through the suction channel 112b to provide continuous fluid recirculation during the fluidic massage. In one embodiment, the main control unit 111 may be configured to initially supply fluid to the bladder 103 through the supply channel 112a until a predetermined volume of fluid accumulates within the bladder 103 prior to initiating suction through the suction channel 112b. Once the predetermined volume has accumulated, the at least one pump 114 simultaneously supplies additional fluid while withdrawing excess fluid, thereby maintaining a substantially constant fluid volume within the bladder 103 during therapeutic operation. The continuous fluid recirculation generates periodic expansion and contraction of the bladder 103, transmitting pulsating pressure and fluidic massage to the body of the user in contact with the outer surface of the bladder 103.

The main control unit 111 may selectively adjust one or more operational parameters, including flow rate, suction pressure, pulsation frequency, massage intensity, treatment duration, or combinations thereof, in response to user inputs received through one or more push buttons 108, a touch interface, a wireless communication module, or other input mechanisms. In various embodiments, the ball-shaped fluidic massage device 1000 may further comprise one or more radiation sources 207 provided on or integrated with the bladder 103 for emitting therapeutic electromagnetic radiation toward the body of the user while the bladder 103 simultaneously provides fluidic massage. The bladder 103 may be fabricated from a flexible transparent, translucent, or optically transmissive material to permit passage of electromagnetic radiation emitted by the one or more radiation sources 207. toward the treatment region, thereby enabling concurrent phototherapy and fluidic massage treatment.

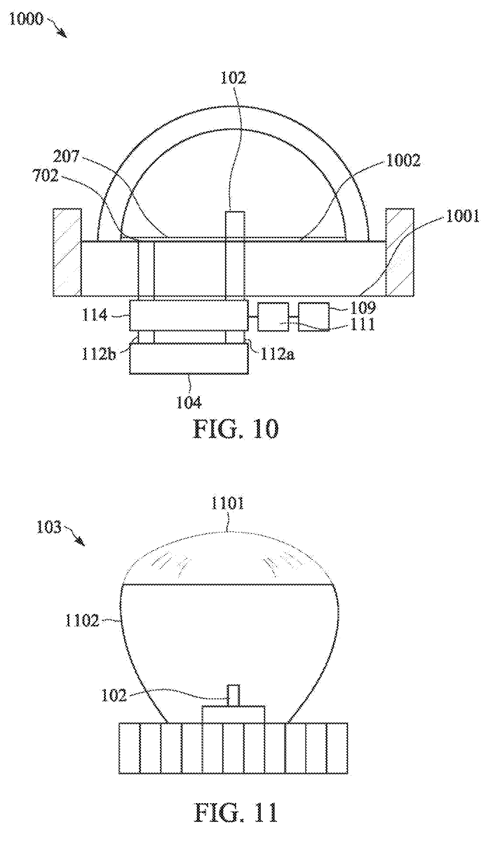

Referring to FIG. 11, in yet another embodiment, a bladder configuration is illustrated wherein the bladder 103 comprises a contact portion 1101 and one or more side walls 1102 having different material properties. The contact portion 1101 is configured to contact the skin of the user during therapeutic use and is fabricated from a flexible material including flexible polymer materials, elastomeric materials, silicone-based materials, or fabric materials. The flexible contact portion 1101 comfortably conforms to the contour of the body portion being treated and effectively transmits the fluidic massage sensation generated by the impinging massage medium. The one or more side walls 1102 are fabricated from a substantially rigid or semi-rigid material including polymeric materials, thermoplastic materials, or composite materials. The rigid side walls 1102 maintain the structural integrity of the bladder 103 during operation and prevent the bladder 103 from becoming flaccid when the massage medium is withdrawn through the suction channel (fluidic channel 112b). This configuration ensures consistent bladder geometry and massage performance throughout the treatment cycle. The bladder configuration illustrated in FIG. 11 may be applied to any of the embodiments described herein, including handheld devices, wearable therapy pads, wearable therapy caps, ball-shaped devices, or other configurations.

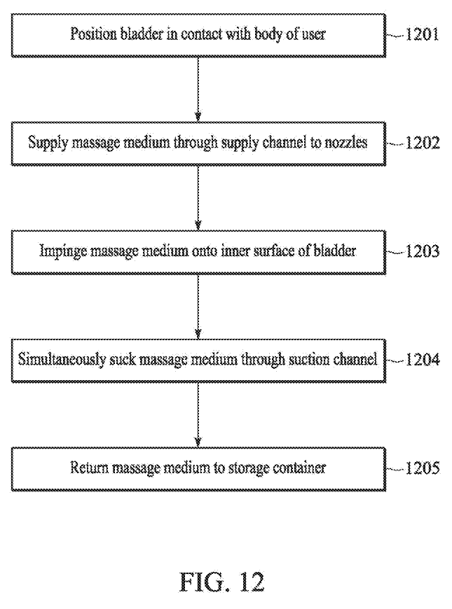

Referring to FIG. 12, a flowchart 1200 depicts a method for providing fluidic massage to a user, in accordance with an embodiment of the present invention. The method begins at step 1201, wherein a bladder is positioned in contact with a body of the user such that an outer surface of the bladder contacts the body and one or more nozzles are encapsulated within the bladder. At step 1202, a massage medium is supplied from a storage container through a supply channel to the one or more nozzles. At step 1203, the massage medium is impinged from the one or more nozzles in one or more patterns onto an inner surface of the bladder to provide fluidic massage to the user through the bladder. At step 1204, the massage medium is simultaneously sucked from the bladder through a suction channel. At step 1205, the massage medium is returned to the storage container to provide continuous recirculation during the fluidic massage. The method may optionally include additional steps such as accumulating a predetermined volume of massage medium within the bladder prior to initiating suction, heating or cooling the massage medium prior to supply, emitting therapeutic electromagnetic radiation through the bladder, or delivering electrical stimulation to the user in conjunction with the fluidic massage.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A device for providing fluidic massage, the device comprising:
a storage container adapted to store a massage medium;
a plurality of fluidic channels including a supply channel and a suction channel;
at least one pump in fluidic communication with the storage container through the plurality of fluidic channels;
a main control unit configured to control operation of the at least one pump;
one or more nozzles configured to impinge the massage medium in one or more patterns on an inner surface of a bladder to provide fluidic massage to a user through the bladder, wherein an outer surface of the bladder is adapted to be in contact with the user; and
wherein the device is configured to simultaneously supply the massage medium to the bladder through the supply channel and suck the massage medium from the bladder through the suction channel to provide continuous recirculation during the fluidic massage.

2. The device of claim 1, wherein the massage medium comprises one or more of water, a therapeutic fluid, an emulsion, essential oils, or air.

3. The device of claim 1, wherein the device is configured to initially supply the massage medium to the bladder through the supply channel until a predetermined volume accumulates within the bladder prior to initiating suction through the suction channel.

4. The device of claim 1, further comprising a detachable bladder assembly configured to be removably coupled to a housing of the device, the detachable bladder assembly comprising the bladder secured to an attachment rim, the attachment rim configured to releasably engage the housing through one or more locking mechanisms.

5. The device of claim 1, further comprising one or more radiation sources provided on or integrated with the bladder, the one or more radiation sources configured to emit therapeutic electromagnetic radiation toward the body of the user while the bladder simultaneously provides fluidic massage.

6. The device of claim 5, wherein the bladder is fabricated from a flexible transparent, translucent, or optically transmissive material through which electromagnetic radiation emitted by the one or more radiation sources passes toward the body of the user.

7. The device of claim 1, wherein the bladder comprises a contact portion and one or more side walls, wherein the contact portion configured to contact the skin of the user is fabricated from a flexible material, and wherein the one or more side walls are fabricated from a substantially rigid material to maintain structural integrity of the bladder during operation.

8. The device of claim 1, wherein the device is configured as a wearable therapy pad comprising a flexible pad body adapted to be removably worn over a selected portion of the body of the user, one or more fastening members configured to removably secure the wearable therapy pad around the selected body portion, and a plurality of bladders distributed across a body-contacting surface of the flexible pad body.

9. The device of claim 8, wherein the plurality of bladders are arranged in a plurality of distinct zones, wherein the main control unit is configured to independently control massage medium supply and suction to each zone to provide zone-based massage including sequential activation, pulse-based massage, or wave-like massage patterns across different zones.

10. The device of claim 1, wherein the device is configured as a wearable therapy cap comprising a flexible cap body adapted to surround at least a portion of a head of the user, one or more adjustable fastening members for accommodating different head sizes, and a plurality of bladders distributed over an inner surface of the therapy cap.

11. The device of claim 1, wherein the device comprises a ball-shaped body having an inner layer and an outer layer, wherein the inner layer houses the storage container, the at least one pump, and the main control unit, and wherein the outer layer comprises the bladder affixed to an exterior of the inner layer.

12. A fluidic massage system comprising:
a massage delivery unit comprising one or more bladders, each bladder having an outer surface adapted to contact a body of a user and an inner surface, and one or more nozzles configured to impinge a massage medium in one or more patterns on the inner surface of the one or more bladders to provide fluidic massage to the user through the one or more bladders;
a storage unit comprising a storage container adapted to store the massage medium;
a pump unit comprising at least one pump configured to circulate the massage medium;
a plurality of fluidic channels including a supply channel and a suction channel fluidically connecting the storage unit, the pump unit, and the massage delivery unit;
a control unit configured to control operation of the at least one pump; and
wherein the system is configured to simultaneously supply the massage medium to the one or more bladders through the supply channel and suck the massage medium from the one or more bladders through the suction channel to provide continuous recirculation during the fluidic massage.

13. The fluidic massage system of claim 12, wherein the pump unit is positioned internal to the massage delivery unit.

14. The fluidic massage system of claim 12, wherein the pump unit is positioned external to the massage delivery unit and fluidically connected thereto through one or more flexible conduits.

15. The fluidic massage system of claim 12, wherein the storage unit is positioned internal to the massage delivery unit.

16. The fluidic massage system of claim 12, wherein the storage unit is positioned external to the massage delivery unit and fluidically connected thereto through one or more flexible conduits.

17. The fluidic massage system of claim 12, further comprising a temperature control unit configured to heat or cool the massage medium for providing heat therapy or cold therapy in conjunction with the fluidic massage.

18. The fluidic massage system of claim 12, further comprising one or more electrical stimulation elements configured to deliver transcutaneous electrical nerve stimulation (TENS) or electrical muscle stimulation (EMS) to the user in conjunction with the fluidic massage.

19. A method for providing fluidic massage to a user, the method comprising:
positioning a bladder in contact with a body of the user, wherein an outer surface of the bladder contacts the body and one or more nozzles are encapsulated within the bladder;
supplying a massage medium from a storage container through a supply channel to the one or more nozzles;
impinging the massage medium from the one or more nozzles in one or more patterns onto an inner surface of the bladder to provide fluidic massage to the user through the bladder;
simultaneously sucking the massage medium from the bladder through a suction channel; and
returning the massage medium to the storage container to provide continuous recirculation during the fluidic massage.

20. The method of claim 19, further comprising one or more of heating or cooling the massage medium prior to supply, emitting therapeutic electromagnetic radiation through the bladder, or delivering electrical stimulation to the user in conjunction with the fluidic massage.
